(19) United States
(12) Patent Application Publication
(10) Pub. No.: US 2021/0315588 A1
(43) Pub. Date: Oct. 14, 2021

(54) First Responder Bracelet

(71) Applicant: Alvin Joseph Jolivette, JR., Huntington Beach, CA (US)

(72) Inventor: Alvin Joseph Jolivette, JR., Huntington Beach, CA (US)

(21) Appl. No.: 17/229,609

(22) Filed: Apr. 13, 2021

Related U.S. Application Data
(60) Provisional application No. 63/009,393, filed on Apr. 13, 2020.

(51) Int. Cl. A61B 17/132

(52) U.S. Cl. CPC A61B 17/1327

(57) ABSTRACT

The present invention relates to a first responder bracelet that includes a sleeve and a tassel both connected at one end and another end engages when user wears it. The bracelet unfolds into a tourniquet. The tourniquet includes a strap within a sleeve. The strap is fixed with a strap lock with a buckle at one end. Additionally, a twist lock is fixed to the strap at the other end. The twist lock is capable of twisting and engaging with the strap lock near the buckle area.

### CLAIM TO PRIORITY

This application claims priority to U.S. provisional application 63/009,393 filed on 13 Apr. 2020 and is hereby incorporated into this specification by reference for all that it discloses for all purposes.

### TITLE

First Responder Bracelet

### FIELD OF INVENTION

The invention relates to a First Responder Bracelet. More specifically, the present invention provides a secure first responder bracelet configured to be converted to a tourniquet and a novel method to wear it.

### BACKGROUND OF THE INVENTION

A traditional tourniquet is made from a stick and a rope (or leather belt). The rope of the tourniquet is made into a loop that fits over the damaged limb, and the stick is inserted through the loop. The loop is then tightened by twisting the stick. This may limit the flow of blood with side effects, including soft tissue damage and sometimes nerve damage.

Initially, First Responder Bracelets were used by the firefighters or emergency evacuators, or rescuers for labeling the patients according to their medical condition. The color of the bracelet would be different for different medical conditions. So, when they reach the hospital, they receive medical care in accordance with the need.

During the fourth century, tourniquets were used by the wounded soldiers to stop the flow of blood. These tourniquets were narrow straps made up of bronze and used only leather for comfort. The narrow straps further caused uneven friction or pressure through the strap and provided less coverage area.

Currently, the tourniquets present in the market are silicone ring tourniquets or elastic ring tourniquets. These tourniquets mainly consist of a silicone ring, stockinet, and pull straps made from ribbon, which are used to roll the device onto the limb. The surgical tourniquets enable the surgeons to work in a bloodless operative field by preventing blood flow to a limb and allow surgical procedures to be performed with improved accuracy, safety, and speed. The use of tourniquets during upper-extremity surgery involves a common complication like nerve or muscle injury.

However, the currently available tourniquets are more improved and advanced. But, these tourniquets still are not capable of avoiding friction between the strap and the limb that creates a region with low pressure where tension is not evenly distributed through the strap. The strap around the limb's circumference causes the arterial blood not to be completely staunched despite high tension on the strap.

In general, the application of uneven pressure around the limb may drive overall tourniquet pressures even much higher to stop arterial blood flow while increasing the probability of injuries to nerves, muscles, and limbs. These injuries are common for those engaged in recreational activities or in armed forces where the risk of serious injury is increased. Furthermore, a tourniquet that is too bulky or has an excessive weight would not be considered for packing and carrying by the potential user. Additionally, a small, lightweight tourniquet can be easily packed and included as a part of recreational gear.

Hence, there is a need for a lightweight, easy to use, a tourniquet that effectively restricts blood flow. Additionally, the tourniquet shall be easily applied with one hand without pinching, tearing, or otherwise damaging soft tissues.

### SUMMARY OF THE INVENTION

An objective of the present invention is to provide an improved and secured tourniquet. A tourniquet, also called a first responder bracelet, is a device that applies pressure to a limb or extremity to limit but not to stop the flow of blood. The tourniquet is a circular system, including a sleeve and a strap. Furthermore, the strap includes a twist-lock and a strap lock with a buckle. The twist-lock twists and further engages with a strap lock. The sleeve further includes two slits at opposite ends for the passage of the twist lock and the strap lock outwards.

The strap with elastic properties in a twisted state is inserted within the sleeve. The ring-shaped sleeve is provided with two slits on the opposite ends. Herein, the twist lock extends outward from the sleeve through one slit while the strap lock extends outward from the other slit. The ring-shaped sleeve could be used on an arm, chest, legs, or else. The twist lock is stretched and locks the strap tight on the arm. As the twist lock stretches, so does the stretchable strap, as they both are in connection with each other. Furthermore, the twist lock is twisted to the point where circulation cuts off and then inserted into the strap lock to secure it properly at a particular place.

The other objective of the present invention is to provide pulling and twisting of the twist lock. Herein, the twist lock with the buckle is pushed to secure the strap on a user's arm. As the twist lock stretches, so does the stretchable strap, as they both are in connection with each other. Furthermore, the twist lock is twisted to the point where circulation cuts off and then inserted into the strap lock to secure it properly at a particular place.

A component of the secured tourniquet is a sleeve member. The stretchable strap is placed within the sleeve. When the user applies a force to tighten the tourniquet, a buckle near the end of the strap is drawn towards the sleeve. Once the pressure is adequately applied over the arm, the tourniquet is secured in a particular position through the twist lock being twisted and secured in the twist strap lock. Further, resulting in an even application of pressure, without any blood loss or any other damage caused to the nerve or muscles.

Other objectives and aspects of the invention are to provide individuals with life-saving tools at all times, designed in the form of a bracelet. The tourniquet is folded and twisted very tightly to form a bracelet. The bracelet includes a tassel that is used to latch the twisting handle after it is turned tight. The bracelet is unfoldable and made from a strong yet thin stretchable type fabric. It has a loop at the end to feed through the other end so that it can be pulled tight on a limb before twisting to full tightness. The twisting lever is in the form of custom needs that connect into each other to form a small rod used for twisting the bracelet tight. After the rod is twisted to its maximum tightness, the tassel slides down to lock it in. Such a bracelet can be used to treat trauma and save lives.

Yet other objectives and aspects of the invention will become apparent from the following detailed description, taken in conjunction with the accompanying drawings, which illustrate, by way of example, the features in accordance with embodiments of the invention.

Embodiments of the present invention may employ any or all of the exemplary aspects above. Those skilled in the art will further appreciate the above-noted features and advantages of the invention together with other important aspects thereof upon reading the detailed description that follows in conjunction with the drawings, which illustrate, by way of example, the features in accordance with embodiments of the invention. The summary is not intended to limit the scope of the invention, which is defined solely by the claims attached hereto.

### BRIEF DESCRIPTION OF THE DRAWINGS

Embodiments of the invention are described with reference to the following drawings, wherein:

FIG. 1 illustrates a perspective view of a first responding bracelet;

FIG. 2 illustrates a view of the unfolding of the first responding bracelet to a tourniquet;

FIG. 3 illustrates a perspective view of the tourniquet in another embodiment;

FIG. 4 illustrates an exploded view of the tourniquet;

FIG. 5a illustrates a view of the tourniquet in a normal resting position;

FIG. 5b illustrates a view of the tourniquet in a stretched position;

FIG. 5c illustrates a view of the tourniquet in a locked position;

FIG. 6 illustrates a view of the tourniquet on a wrist; and

FIG. 7 illustrates a method of wearing the tourniquet on a body part of a user.

### DETAILED DESCRIPTION OF THE DRAWINGS

The present invention relates to a First Responder Bracelet that unfolds into a Tourniquet, a device for stopping the flow of blood through a vein or an artery. Specifically, the present invention relates to a modified first responder bracelet that a user can wear regularly and, during an emergency, can be used as a tourniquet, an AED, or a tracheotomy kit. The tourniquet is folded and twisted very tightly to form a bracelet. The bracelet also includes a tassel that is used to latch the twisting handle after it is turned tight. When unfolded, the bracelet turns to a tourniquet. The tourniquet includes a sleeve and a strap. The strap comprises a twist lock and a strap lock with a buckle. The sleeve is provided with a pair of slits. The twist lock twists and further engages in the strap lock along the buckle area. Moreover, the sleeve also includes two slits on the opposite ends for the exit of the buckle and the twist lock, respectively. The tourniquet is lightweight and easy to use, particularly for travelers and people in the armed forces.

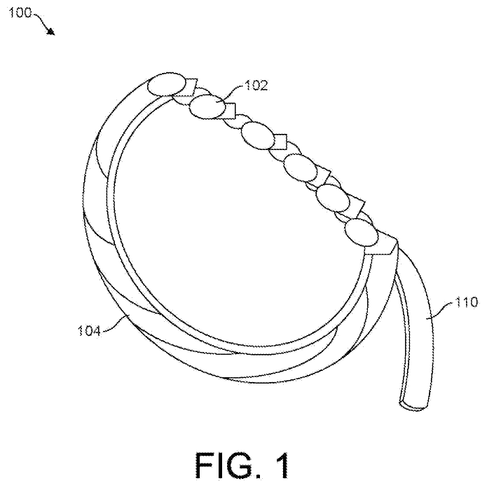

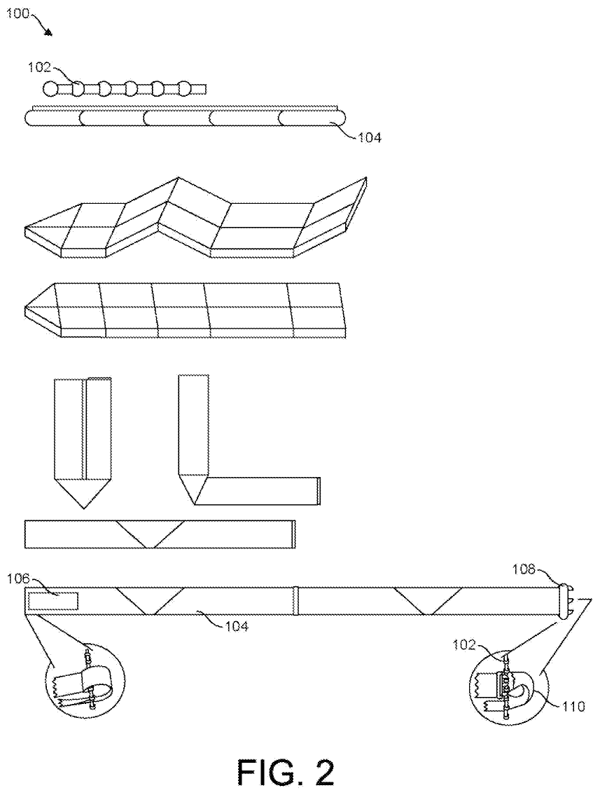

FIG. 1 illustrates a perspective view of a first responding bracelet with may also be referred to as a convertible bracelet. The invention provides individuals with life-saving tools designed in the form of a bracelet 100. The bracelet in the perspective view is in a folded stage. That is folded into many folds both vertically and horizontally and twisted very tightly. The bracelet also includes a tassel 110 that is used to latch the twisting handle 104 after it is turned tight. The first responder bracelet comprises a thin material that stretches to more than 18″. It has a loop 106 (FIG. 2) at the end to feed through the other end so that it can be pulled tight on a limb before twisting to full tightness. In a completely folded configuration, one end of the bracelet includes a receiving end 108, which lies in the center in an open configuration. The receiving end 108 is there to hold or receive the tassel 110 when a user wears it. The tassel includes two parts, a beaded part 102 and a tuft 110 or a plume like part. Both the ends of the tassel engage with each end of the bracelet's band 104. The bracelet 100 also includes a twisting lever 112 that can be in different forms, including different shapes and sizes that connect to form a small rod 112 used to twist the tourniquet. The beads 102 and the tassel may all be the same color. Alternatively, one or more bead(s) may be of different colors depicting a patient's condition, wherein one color is for one kind of condition. For example, a first color could indicate a patent has been vaccinated for a disease such as COVID-19, while a second color may indicate a patent has COVID-19, and a third color may indicate an untested patient with COVID-19 symptoms. Any disease may replace the disease “COVID-19” in the previous example.

FIG. 2 illustrates a view depicting the unfolding of the first responding bracelet into a tourniquet. The bracelet 100 may be folded into many folds both vertically and horizontally and twisted very tightly. Also attached with the bracelet is a tassel 110 used to latch the twisting handle 104 after it is turned tight. As the bracelet 100 is unfolded into a tourniquet, the strong yet thin material, made of stretchable fabric, is presented. It has a loop 106 at the end to feed through the other end so that it can be pulled tight on a body part before twisting to full tightness. The tassel 110 my further define a twisting lever that can be in different forms, including different shapes and sizes that connect into each other to form a small rod used for twisting the tourniquet tight. After the twisting lever is twisted to its maximum tightness. The tassel 110 finally slides down to lock it in. These kinds of bracelets can be used to treat trauma and are portable.

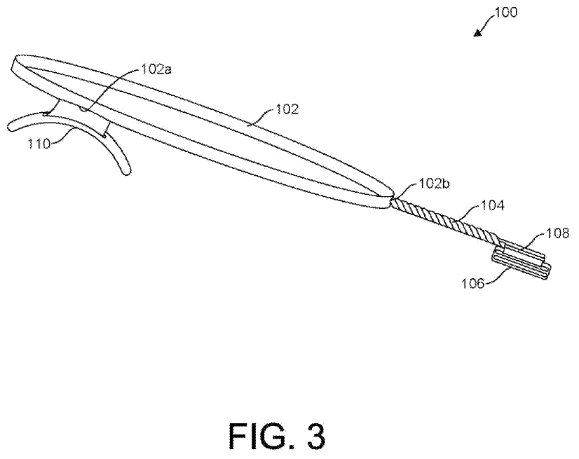

FIG. 3 illustrates a perspective view of a tourniquet in another embodiment. The tourniquet 100 includes a sleeve 102 and a strap 104. The strap 104 in a twisted state is embedded within the sleeve 102 through the pair of slits (102a, 102b) disposed on opposite ends of the strap. The strap 104 comprises a buckle 106, a strap lock 108, and a twist lock 110. Moreover, the sleeve 110 is provided with a pair of slits (102a, 102b).

The buckle 106 and the twist lock 110 extend out from each of the pair of slits (102a, 102b) within the sleeve 102. The twist lock 110 twists and further engages with a strap lock 108. Furthermore, the twist lock 110 is twisted to the point where circulation cuts off and then inserted into the strap lock 108 to secure it properly at a particular place. The connection of the twist lock 110 with the strap 104 is established via an opening in the twist lock 110 through which the strap 104 passes. In a similar manner, the connection of the buckle 106 and the strap 104 is achieved. Also, the strap lock 108 communicates both with the strap 104 and the buckle 106.

The strap 104 with elastic properties in a twisted state is inserted within the sleeve 102. The ring shaped sleeve 102 is provided with two slits (102a, 102b) on the opposite ends. The ring shaped sleeve 102 could be used on an arm, the chest, the legs other experiments.

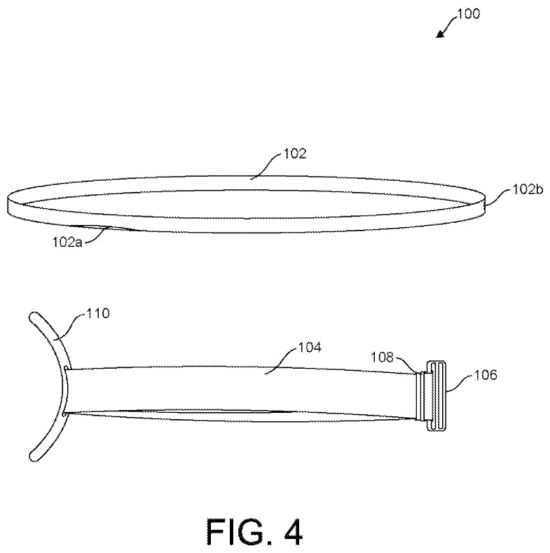

FIG. 4 illustrates an exploded view of the tourniquet 100. The exploded view shows the sleeve 102 and the strap 104 separately. The sleeve 102 comprises two slits (102a, 102b), and the strap 104 comprises a buckle 106, a strap lock 108, and a twist lock 110. The strap 104 is shown in an untwisted form. The untwisted form occurs only when the sleeve 102 and strap 104 are separate from one another. When the strap and sleeve assembly are combined, the strap 104 exists in a twisted form.

The sleeve 102 may be ring shaped with a pair of slits (102a, 102b) within it. One slit 102a, out of the pair of slits, may be provided for the exit of the twist lock 110, while the other slit 102b remains at some distance from the initial one. The other slit 102b is used for the exit of the strap lock 108 along with the buckle 106. The sleeve 102 may be composed of a sterile fine nylon fabric. Alternatively, the sleeve 102 may be of any latex-free fabric, non-woven fabric, or any suitable fabric.

The strap 104, in an exploded configuration, occurs in an untwisted form. The strap 104 is a stretchable material like an elastic band, abs plastic, or any suitable material. In another embodiment, the strap 104 may be a woven fabric or a latex fabric, or similar material. The strap 104 further includes a twist lock 110, a buckle 106, and a strap lock 108. The connection between the twist lock 110 with the strap 104 may be established via an opening in the twist lock 110 through which the strap 104 passes. In a similar manner, a connection between a buckle 106 and a strap 104 is achieved. Also, the strap lock 108 communicates both with the strap 104 and the buckle 106. The buckle 106 and the twist lock 110 are of hard plastic, and the strap lock 108 may be of the same material as the strap 104.

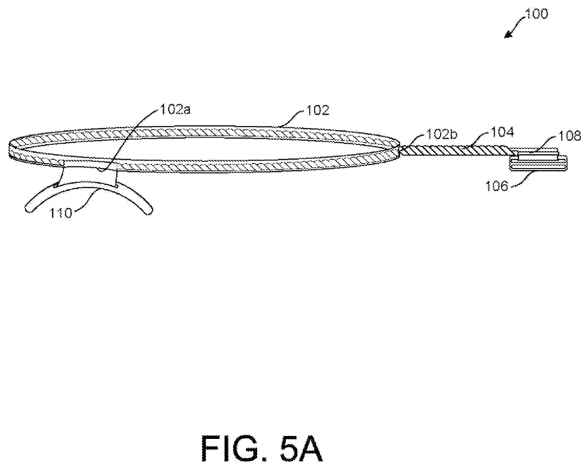

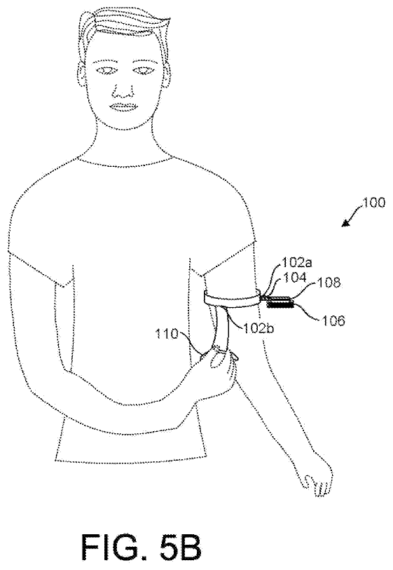

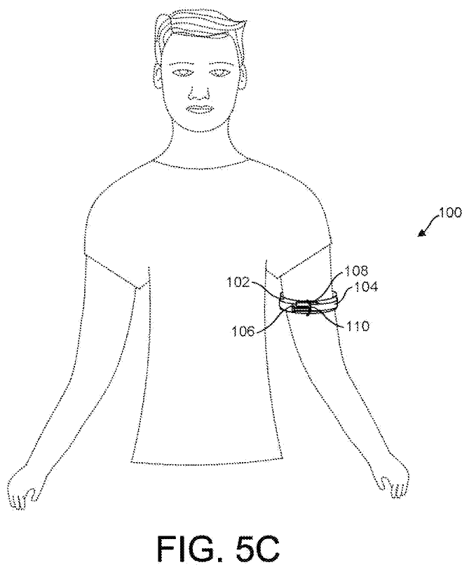

FIG. 5+a illustrates a view of the tourniquet 100 in a normal resting position. The stretchable strap 104 in a twisted form may be positioned within the sleeve 102. The twist lock 110 may be directly engaged on one end of the sleeve 102 through the slit 102a, while strap lock 108 and the buckle 106 are provided at the other end of the sleeve 102 through another slit 102b. Furthermore, the buckle 106 and the twist lock 110 are not directly in touch with the sleeve 102 but are at some distant position with the parts of the strap 104. So, the twist lock 110 and the buckle 106 are in a resting position when not in use.

FIG. 5b illustrates a view of the tourniquet 100 in a stretched position. The stretchable strap 104 in a twisted form may be positioned within the sleeve 102. When the user applies force to tighten the tourniquet 100, the end of the strap 104 nearby buckle 106 may be drawn towards the sleeve 102. A force may be applied from the terminal point by pulling the twist lock 110. The movement of the strap 104 inside the sleeve 102 should be smooth, so any damage to the user is minimized. In a stretched position, the strap lock 108 and the buckle 106 are directly engaged on one end of the sleeve 102, while the twist lock 110 is provided at the other terminal of the sleeve 102. Furthermore, the twist lock 110 should not be in direct contact with the sleeve 102 but maybe be at some distant position with the rest part of the strap 102. The material of the sleeve 102 is of soft and sterile fabric and of larger width to cover a larger surface area, thereby providing a more supportive and smooth tightening of the sleeve 102 of the tourniquet 100 on the user's arm.

FIG. 5c illustrates a view of the tourniquet 100 in a locked position. The stretchable strap 104, in a twisted form, maybe be positioned within the sleeve 102. The sleeve 102 may provide a pair of slits (102a, 102b). The twist lock 110 twists and further engages with the strap lock 108. While stretching, the buckle 106 comes to a position closer to the other slit at the end of the sleeve 102, wherein the twist lock 110 engages with the strap lock 108. The locking process is considered adequate when the pressure is correctly applied over the arm, and the tourniquet 100 is secured in a particular position through the twist lock 110 being twisted and inserted in the strap lock 108. The twist lock 110 is twisted to the point where blood circulation cuts off. Afterward, the twist lock 110 may be inserted into the strap lock 108 to lock the tourniquet 100 at a particular position.

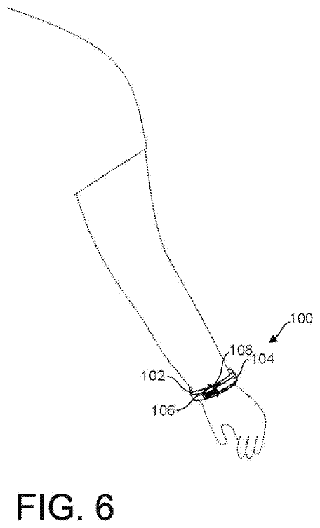

FIG. 6 illustrates a view of the tourniquet on a user's wrist. The tourniquet 100 on a wrist 112 includes a sleeve 102 and a strap 104. The sleeve 102 may be ring shaped or any suitable bracelet shape. The strap 104 comprises a buckle 106, a strap lock 108, and a twist lock 110. Moreover, the sleeve 110 may be provided with a pair of slits (102a, 102b). The buckle 106 and the twist lock 110 extend out from each of the pair of slits (102a, 102b) within the sleeve 102. Wherein, the twist lock 110 twists and further engages with a strap lock 108. Furthermore, the twist lock 110 may be twisted to the point where circulation to the wrist 112 is cut off. The twist lock may then be secured in the strap lock 108 to secure it properly at the wrist 112.

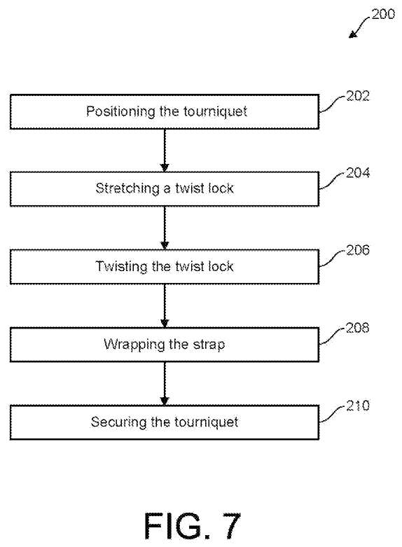

FIG. 7 illustrates the steps of associating a bracelet with a body part. A method of wearing a tourniquet 200 comprises the positioning of the tourniquet 202 near the injured area. Stretching the bracelet 204, wherein the stretching includes pulling the twist lock in the outward direction through one slit, causing the strap to stretch. Further, the pull directs the pushing of a strap lock with a buckle towards the other slit. The next step is to twist the twist lock 206 to a point where circulation cuts off. Next, the strap 208 is stretched around the body part. The bracelet 210 is secured in a locked position, wherein the twist lock is engaged with the strap lock.

Although the invention is described above in terms of various exemplary embodiments and implementations, it should be understood that the various features, aspects, and functionality described in one or more of the individual embodiments are not limited in their applicability to the particular embodiment with which they are described, but instead can be applied, alone or in various combinations, to one or more of the other embodiments of the invention, whether or not such embodiments are described and whether or not such features are presented as being a part of a described embodiment. Thus, the present invention's breadth and scope should not be limited by any of the above-described exemplary embodiments.

The presence of broadening words and phrases such as “one or more,” “at least,” “but not limited to” or other like phrases in some instances shall not be read to mean that the narrower case is intended or required in instances where such broadening phrases may be absent.

1. A tourniquet comprising:
a sleeve defining a first slit and a second slit;
a strap removably disposed within said sleeve, said strap defining a first strap end and an opposing second strap end, said strap further comprising:
a twist lock associated with said first strap end wherein said twist lock extends outward from said sleeve through the first slit; and
a strap lock defining a buckle associated with said second strap end wherein said strap lock extends outward from said sleeve through the second slit.

2. A tourniquet as in claim 1, wherein the tourniquet defines a convertible bracelet wherein said convertible bracelet defines a bracelet when said strap is disposed inside said sleeve and wherein said convertible bracelet defines a tourniquet when said strap is removed from said sleeve.

3. A method to wear a tourniquet, the method comprising:
positioning the tourniquet on a body part of a user, wherein the tourniquet includes a strap within a sleeve;
stretching a twist lock fixed at one end of the strap, wherein the twist lock is stretched as per the user comfort;
twisting the twist lock;
wrapping the strap around the body part; and
securing the tourniquet by a strap lock fixed at other end of the strap, wherein the securing is achieved by engaging the strap lock with the twist lock.

4. A first responding bracelet comprising:
a tassel with a first part and a second part, wherein the first part includes multiple beads and the second part includes a tuft; and
a sleeve including
a loop;
a receiving unit;
a twisting lever; and
a twisting handle, wherein the loop at end feeds through another end, so that it can be pulled tight on a body part through twisting handle; further wherein, the twisting handle is locked at a position through the tassel.
